(19) United States
(12) Patent Application Publication
(10) Pub. No.: US 2026/0103699 A1
(43) Pub. Date: Apr. 16, 2026

(54) Capture And Selective Release Of Biological Material

(71) Applicant: Illumina, Inc., San Diego, CA (US)

(72) Inventors: Aaron Aslanian, San Diego, CA (US); Jian-Sen Li, San Diego, CA (US); Ali Asadi, La Jolla, CA (US); Xin Sheng, San Diego, CA (US)

(21) Appl. No.: 19/420,096

(22) Filed: Dec. 15, 2025

Related U.S. Application Data
(63) Continuation of application No. PCT/US2024/048462, filed on Sep. 25, 2024.
(60) Provisional application No. 63/586,180, filed on Sep. 28, 2023.

(51) Int. Cl. C12N 15/10

(52) U.S. Cl. CPC C12N 15/101

(57) ABSTRACT

A biological material complex is described herein. In some embodiments, the biological material complex includes a solid surface having a terminal hydrazine that is available to bind to a functional group of a biological material. Applications, uses, and variations of the disclosed complex include, but are not limited to, separating, purifying, and labeling biological materials.

### CROSS REFERENCE TO RELATED APPLICATIONS

This application is a continuation application claiming priority to PCT/US24/48462, which claims priority from and the benefit of U.S. Provisional Application Ser. No. 63/586,180, entitled “CAPTURE AND SELECTIVE RELEASE OF BIOLOGICAL MATERIAL”, filed Sep. 28, 2023, which is hereby incorporated by reference in its entirety for all purposes.

### BACKGROUND

The technology disclosed relates to separation and purification of biological material. In particular, the technology disclosed relates to capture and selective release of biological material using a capture complex bound to a solid substrate.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Purification of biological material may include capturing the biological material using a substrate, removing the biological material from other impurities, and releasing the biological material from the substrate and into a target location with fewer impurities. However, the properties that make a substrate effective at capturing the biological material may make it difficult to release the biological material. For example, certain substrates may link or bond to the biological material in a permanent or semi-permanent manner, making releasing the biological material difficult. Further, substrates do not form as permanent of a link or bond to the biological material may not capture a relatively large percentage of the biological material, thereby potentially wasting large amounts of expensive material. Accordingly, it is desirable to develop techniques that may efficiently capture biological materials and, if desired, enable selective release of the biological materials.

### BRIEF DESCRIPTION

In one embodiment, the present disclosure relates to a biological material complex. The biological material complex includes a solid surface. Additionally, the biological material complex includes a linker coupled to the solid surface at a first end of the linker and a terminal hydrazine or hydrazide coupled to a second end of the linker.

In another embodiment, the present disclosure relates to a method of preparing a biological material complex. The method includes hybridizing a first end of a linker to a solid surface to form a biological material substrate, and the linker includes a terminal hydrazine at a second end of the linker. The method also includes providing a biological material to a volume including the biological material substrate, wherein the biological material substrate comprises a terminal aldehyde. Further, the method includes linking the biological material and the biological material substrate via a reaction between the terminal hydrazine and the terminal aldehyde.

In another embodiment, the present disclosure relates to a linked biological material complex. The linked biological material complex includes a solid surface. The linked biological material complex also includes a linker coupled to the solid surface at a first end of the linker and a terminal hydrazine coupled to a second end of the linker, and wherein the linker comprises an electron withdrawing group. Further, the linked biological material complex includes a biological material coupled to the second end of the linker via a hydrazone, wherein the hydrazone is proximate to the electron withdrawing group.

The preceding description is presented to enable the making and use of the technology disclosed. Various modifications to the disclosed implementations will be apparent, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein. The scope of the technology disclosed is defined by the appended claims.

### BRIEF DESCRIPTION OF THE DRAWINGS

These and other features, aspects, and advantages of the present techniques will become better understood when the following detailed description is read with reference to the accompanying drawings in which like characters represent like parts throughout the drawings, wherein:

FIG. 1 is schematic diagram illustrating a biological material complex capturing biological material, in accordance with aspects of the present disclosure;

FIG. 2 is a schematic diagram illustrating the biological material complex of FIG. 1 releasing biological material, in accordance with aspects of the present disclosure;

FIG. 3 shows a method for producing a linker and attaching the linker to a solid surface to form the biological material complex of FIG. 1, in accordance with aspects of the present disclosure;

FIG. 4 shows a method for purifying biological material using the biological material complex of FIG. 1, in accordance with aspects of the present disclosure;

FIG. 5A is a graph showing yield versus pH of a buffer used in the capture of biological material by the biological material complex of FIG. 1, in accordance with aspects of the present disclosure;

FIG. 5B is a graph showing yield versus pH of a buffer used in the release of the biological material from a linked biological material complex, in accordance with aspects of the present disclosure; and

FIG. 6 shows a schematic diagram of a biological material complex with a nickel column solid substrate, in accordance with aspects of the present disclosure.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

As used herein, the term “flow cell” is intended to mean a chamber having a surface across which one or more fluid reagents can be flowed. Generally, a flow cell will have an ingress opening and an egress opening to facilitate flow of fluid. Examples of flow cells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more different nucleic acids can have target nucleotide sequence portions that are different from each other while also having a universal sequence region that is the same for the two or more different nucleic acids.

As used herein, the term “linker” is intended to mean a chemical bond or moiety that covalently bridges two other moieties. A linker can be, for example, the sugar-phosphate backbone that connects nucleotides in a nucleic acid moiety. The linker can include, for example, one or more of a nucleotide moiety, a nucleic acid moiety, a non-nucleotide chemical moiety, a nucleotide analogue moiety, amino acid moiety, polypeptide moiety, or protein moiety. A linker can be non-amplifiable, for example, by virtue of containing a non-nucleic acid moiety. Exemplary linkers are set forth in further detail below and in PCT Pub. No. WO 2012/061832; US Pat. App. Pub. No. 2012/0208724, US Pat. App. Pub. No. 2012/0208705 and PCT App. Ser. No. PCT/US2013/031023, each of which is incorporated herein by reference.

As used herein the term “nucleic acid” can refer to at least two nucleotide monomers linked together. Examples include, but are not limited to DNA, such as genomic or cDNA; RNA, such as mRNA, sRNA or rRNA; or a hybrid of DNA and RNA. As apparent from the examples below and elsewhere herein, a nucleic acid can have a naturally occurring nucleic acid structure or a non-naturally occurring nucleic acid analog structure. A nucleic acid can contain phosphodiester bonds; however, in some embodiments, nucleic acids may have other types of backbones, comprising, for example, phosphoramide, phosphorothioate, phosphorodithioate, O-methylphosphoroamidite and peptide nucleic acid backbones and linkages. Nucleic acids can have positive backbones; non-ionic backbones, and non-ribose based backbones. Nucleic acids may also contain one or more carbocyclic sugars. The nucleic acids used in methods or compositions herein may be single stranded or, alternatively double stranded, as specified. In some embodiments a nucleic acid can contain portions of both double stranded and single stranded sequence, for example, as demonstrated by forked adapters. A nucleic acid can contain any combination of deoxyribo- and ribo-nucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc. In some embodiments, a nucleic acid can include at least one promiscuous base. A promiscuous base can base-pair with more than one different type of base and can be useful, for example, when included in oligonucleotide primers or inserts that are used for random hybridization in complex nucleic acid samples such as genomic DNA samples. An example of a promiscuous base includes inosine that may pair with adenine, thymine, or cytosine. Other examples include hypoxanthine, 5-nitroindole, acylic 5-nitroindole, 4-nitropyrazole, 4-nitroimidazole and 3-nitropyrrole. Promiscuous bases that can base-pair with at least two, three, four or more types of bases can be used.

As used herein, the term “region,” when used in reference to a surface, means an area of the surface that is smaller than the entire area of the surface. The regions can be an area that is smaller than the entire area of a surface that is exposed or accessible to a fluid. Generally the term “region” is used to refer to a continuous, uninterrupted area of a surface, whether or not the region encompasses surface features, sites, contours etc. A region can encompass one or more locations to which a nucleic acid is attached or will be attached.

As used herein, the term “solid support” refers to a rigid substrate that is insoluble in general aqueous liquid. In some embodiments, the “solid support” may be soluble under certain conditions. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. Particularly useful solid supports for some embodiments are located within a flow cell apparatus. Exemplary flow cells are set forth in further detail below.

As used herein, the term “surface,” when used in reference to a material, is intended to mean an external part or external layer of the material. The surface can be in contact with another material such as a gas, liquid, gel, polymer, organic polymer, second surface of a similar or different material, metal, or coat. The surface, or regions thereof, can be substantially flat. The surface can have surface features such as wells, pits, channels, ridges, raised regions, pegs, posts or the like. The material can be, for example, a solid support, gel, or the like.

This disclosure relates to methods and compositions for capturing biological material including, nucleic acids, proteins (e.g., enzymes), peptides, and other types of biological material and, in some instances, releasing the biological material to purifying or isolate biological material. At least in some instances, the disclosed techniques may be used to purify and/or concentrate (e.g., performing both simultaneously) biological material when the starting biological material is relatively low concentration (e.g., less than 10 ppm, less than 1 ppm) to produce a purified product output that is at a relatively high concentration (e.g., 5, 10, 15, 20, 25, 100 times greater than the initial concentration). The disclosed techniques include using a biological material complex that includes substrate (e.g., polymer beads, nanoparticles, flow cells, polystyrene beads, silica beads, metal surfaces) and a linker (e.g., a carbon chain having two or more carbon atoms). The linker is coupled to the substrate (e.g., via a covalent bond) at a first end of the linker, and the linker includes a terminal hydrazine or terminal hydrazide at a second end of the linker. In general, a nitrogen of the hydrazine may perform a nucleophilic attack on the carbon of the aldehyde, thereby forming a hydrazone. In general, it is presently recognized that it may be advantageous to use the chemical transition of a relatively semi-chemically stable first type of bond (e.g., a hydrazone-bond) for catching biological material, while using a relatively more chemically stable (e.g., an oxime-bond) for releasing. In particular, it is presently recognized that it may be advantageous to use a linker that includes a terminal hydrazine that forms a hydrazone after catching the biological material to provide capture and selective release of biological material

In some embodiments, the linker may include an electron withdrawing group (e.g., electron withdrawing functional group, electron withdrawing species, electron withdrawing moiety) that is conjugated, proximate, or adjacent to the terminal hydrazine. As referred to herein, a terminal hydrazine that is “proximate to” an electron withdrawing group refers to when there are, for example, 4, 3, 2, or 1 intervening atoms between an electron withdrawing group and the terminal hydrazine. As referred to herein, a terminal hydrazine that is “adjacent to” an electron withdrawing group refers to when there are 0 intervening atoms between the electron withdrawing group and the terminal hydrazine. As another non-limiting example, in an embodiment when an electron withdrawing group is substituted with one or more terminal hydrazine, the one or more hydrazines may be adjacent to the electron withdrawing group.

In general, the electron withdrawing group may include one or more of amides, sulfonyls, azines (e.g., pyridines, diazines, triazines, and so on), and other electron withdrawing groups known by one of ordinary skill in the art. In any case, the hydrazine-functionalized to the substrate captures the biological material (e.g., forming the resulting hydrazone bond) by reacting with a terminal aldehyde (e.g., a 5′-end aldehyde) of the biological material. In some instances, impurities may be removed from a solution containing the biological material complex through one or more washes, thereby leaving a substrate-captured biological material. At least in some instances, the substrate-captured biological material may be released via temperature, heat, a catalyst, addition of nucleophilic substitution reagent, or a combination thereof. As such, the disclosed techniques may improve the speed of purifying biological material and selectively releasing the biological material.

As discussed above, the disclosed techniques may provide certain advantages, such as concentrating and/or purifying a biological sample. It is also presently recognized that the disclosed techniques may also provide reduce a likelihood of contamination as well as providing shorter turnaround times. As referred to herein, “turnaround times” refer to a time period between producing a product, and subsequently isolating and/or purifying the product.

Certain conventional techniques used to purify biological material include chromatography purification (e.g., high-performance liquid chromatograph (HPLC) of biological material. As compared to HPLC, it is presently recognized that the disclosed techniques may have lower operational costs (e.g., approximately 70%, approximately 75%, approximately 80%, approximately 85%, approximately 90%). For example, chromatography utilizes relatively expensive instruments, such as HPLC columns and, in some instances, dedicated HPLC columns for each type of biological samples to prevent cross contaminations. In contrast, the disclosed techniques may not utilize HPLC instruments. Instead, the disclosed biological material complexes may be readily dispersed into solutions containing the biological material, and thus, it may be easier to integrate the disclosed techniques with existing systems.

Further, and as compared to HPLC, the disclosed techniques may provide a purified biological material product having a purity that is equal to or, in some instance, more pure than would be obtainable by certain conventional purification techniques. For example, HPLC purification for oligonucleotides may provide a small percentage of N-1, N-2 . . . , etc. impurities in the final purified product. These impurities for oligonucleotides may be difficult to remove or otherwise unable to remove from solution with conventional techniques. In contrast, it is presently recognized that the chemical selectively for binding biological material via the terminal hydrazine may substantially prevent, reduce, or eliminate such impurities from oligonucleotides because the hydrazine captures the biological material label (e.g., the 5′-aldehyde label). It should be noted that the 5′-aldehyde label is added during a final step in oligonucleotides synthesis. As such, greater than 99%, greater than 99.9%, or only the full length biological material may include the 5′-aldehyde label, and thus, be captured by the terminal hydrazine of the disclosed linker and selectively released

Further still, the disclosed techniques may provide shorter turnaround time, and therefore much higher throughput. For example, and continuing with a comparison to HPLC, to purify 2000 oligonucleotides purification and assuming 3 HPLC instruments operating 5 days/8 hours, it is estimated that it may take 83 business days to purify the oligonucleotides. In contrast, the disclosed techniques (e.g., catch-release via the linker) may take 14 business days to purify assuming processing 3 of 96 wells plates for each day and 1 day catching/1 day releasing.

Even further, the disclosed techniques may be cross contamination free. For example, the disclosed techniques may be implemented as a disposable device, using disposable solid support and buffers. The HPLC, in contrast, may be dedicated to particular biological samples. Accordingly, the HPLC may be used for multiple different samples (i.e., of the same biological sample) and thus there may be cross-contamination between the different samples. However, the disclosed techniques, at least in some instances, may be implemented as a disposable substrate, and thus, reducing or prevent cross-contamination.

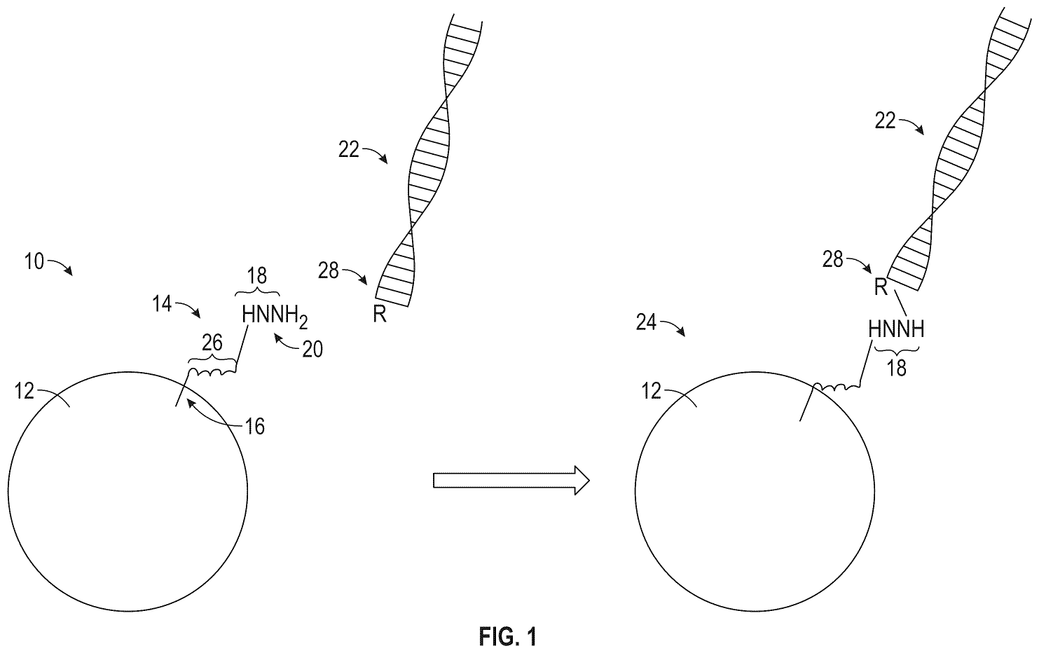

With the foregoing in mind, FIG. 1 shows a schematic diagram of a biological material complex 10. The biological material complex 10 (e.g., biological material substrate, biological material capture complex, biological material capture substrate) generally includes a solid substrate 12 with a linker 14 functionalized, bound, attached, or otherwise coupled to the solid substrate 12 at a first end 16 of the linker 14. The linker 14 includes a terminal hydrazine 18 at a second end 20 of the linker 14. In general, the biological material complex 10 may capture a biological material 22 by reacting with a functional group on the biological material 22 (e.g., an aldehyde), thereby covalently linking (e.g., linking) or covalently bonding (e.g., bonding) to the biological material 22 and forming a linked biological material complex 24.

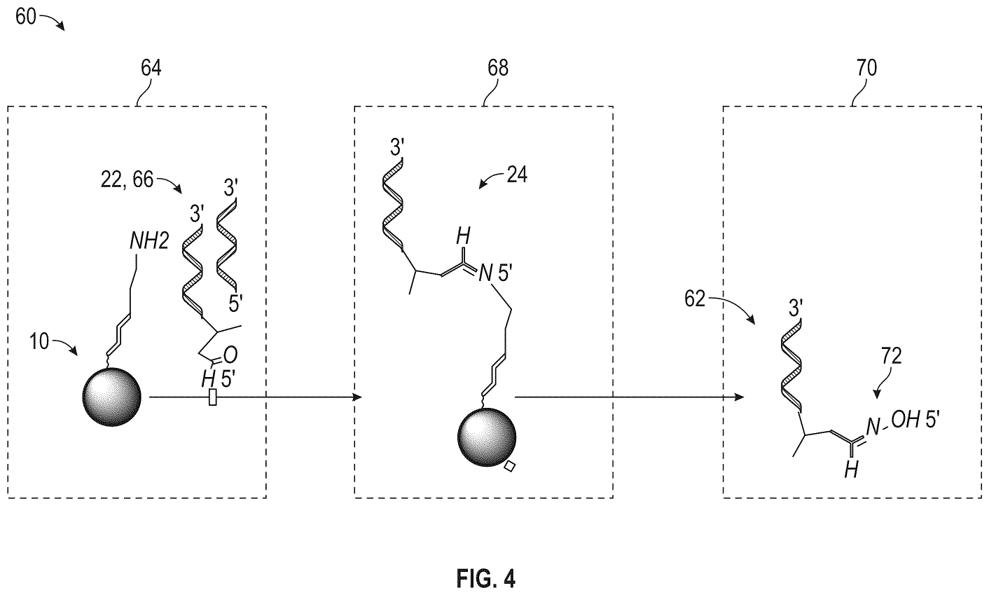

As illustrated, the solid substrate 12 is a bead. For example, the solid substrate 12 may be a polymeric particle, nanoparticle, metal oxide particles, metal oxide nanoparticles, metal nanoparticles, metal particles, polystyrene bead, a silica bead, or a magnetic bead. However, while the solid substrate 12 in the illustrated is a bead, it should be noted that in some embodiments, the solid substrate 12 may be other types of solid surfaces, such as bead chip, flow cells, a nickel column (Ni-column), or substrates. In some embodiments, the solid substrate 12 may be provided as a disposable substrate. In this way, cross contamination may be minimized as the solid substrate 12 is not reused to purify biological material 22 multiple times. Alternatively, the solid substrate 12 may substrate that is reusable by washing the solid substrate 12 after use. It is presently recognized that the disclosed biological material complex 10 may be washable using an acid and/or a base under a controlled condition, as described in more detail with respect to FIG. 4. In some embodiments, the solid substrate 12 may be an outer surface of a portable device that includes linkers 14 functionalized on the outer surface. As such, when the portable device is provided (e.g., dipped) into a solution, the linker 14 may link to the biological material 22, thereby removing the linked biological material 22 from the solution. Accordingly, the linked biological material 22 may be provided to another solution for storage, a reaction, or other use. In some embodiments, the solid substrate 12 may be a surface on a microfluidics device.

In some embodiments, the biological material 22 may include protein or enzymes, such as proteinase K, endonuclease, streptavidin, and polymerase. For example, the linker 14 may capture proteinase K or endonuclease onto the solid substrate 12 via the terminal hydrazine 18. After digest protein or DNA, it may be desirable to remove proteinase K and/or endonuclease. Accordingly, the solid substrate 12 with the linker 14 linked to the proteinase K may be removed from the solution. It is presently recognized that it may be advantageous to use a magnetic solid substrate 12, such as magnetic bead, to facilitate removal of the solid substrate. As another non-limiting example, biological material 22 such as polymerase may be captured by linkers 14 disposed on a solid substrate 12 such as bead chip or flow cell to achieve sequencing-by-synthesis (SBS) chemistry on the bead chip or flow cell surface.

It is presently recognized that fixing an active enzyme onto the solid substrate 12 using the linker 14 may provide certain advantages such as workflow simplification. For example, conventional techniques utilizing proteinase K involve using heat to render the proteinase K inoperable and one or more steps for removal of the proteinase K. These steps may be relatively time-consuming, automation-unfriendly, and may introduce bottlenecks into workflows if desired biological samples are temperature sensitive. However, by fixing proteinase K onto the solid substrate 12 as described above, proteinase K may readily be removed from a solution. Further, fixing the active enzyme onto the solid substrate 12 using the linker 14 may facilitate the re-use of proteinase K. Accordingly, fixing the active enzyme onto the solid substrate 12 may also be low cost (e.g., due to the re-use of proteinase K). Further still, fixing an active enzyme onto the solid substrate 12 using the linker 14 may provide new applications. For example, it is presently recognized that fixing multiple different enzymes (e.g., proteinase K and polymerase) onto the same substrates surfaces may enable one stop shop to perform multiple different reactions. For example, a solid substrate 12 including both proteinase K and polymerase may be capable of removing proteins and amplifying DNA (e.g., simultaneously), which may otherwise not be accomplished when proteinase K and polymerase are free in solution.

The linker 14 includes a molecular chain portion 26 including two or more carbons. For example, the linker 14 may be an alkyl group having 2, 3, 4, 5, 6, 7, 8, or more than 9 carbons between the first end 16 and the second end 20 that includes the terminal hydrazine 18. At the first end 16, the linker 14 may include a suitable functional group for facilitating binding to the solid substrate 12. For example, the functional group at the first end 16 may be an alkoxy group, hydroxyl groups, silanes, phosphates, thiol groups, or other functional groups having a suitable affinity for binding to a solid substrate 12.

In some embodiments, the linker 14 may include an electron withdrawing group along the molecular chain portion 26. For example, the electronic withdrawing group may be an azine, an amide, a phenyl, a sulfonyl, or a combination thereof. It is presently recognized that it may be advantageous to include an electron withdrawing group proximate to or adjacent to, such that there are one or more intervening carbons on the molecular chain between the linker and an electron withdrawing group. For example, without wishing to be bound by theory, it is believed that including an electron withdrawing group adjacent to the terminal hydrazine may stabilize the bond in the resulting hydrazone that forms in the linked biological material complex discussed herein. The biological material 22 may include oligomers, oligonucleotides, enzymes, proteins, and the like.

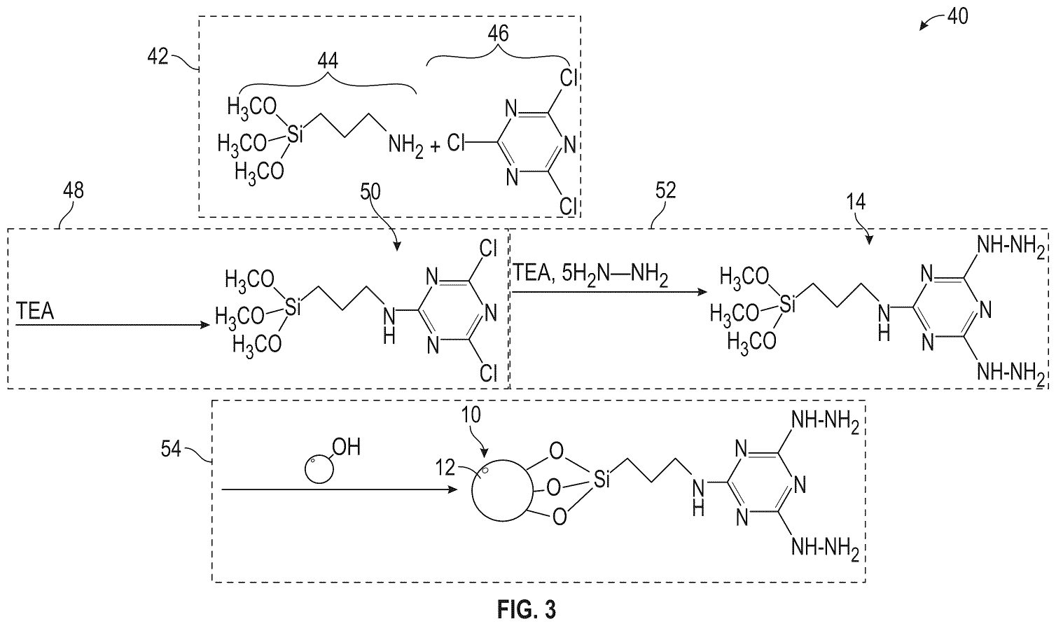

As discussed above, the electron withdrawing group may be an azine. Suitable azines in accordance with present disclosure may include monoazines, diazines, triazines, or a combination thereof. In some embodiments, the electron withdrawing group may include mono-, di-, or tri-substituted monoazines. In some embodiments, the electron withdrawing group may include mono-, di-, or tri-substituted diazines. In some embodiments, the electron withdrawing group may include mono-, di-, or tri-substituted triazines. In some instances, at least one of the functional groups of the substituted azines discussed above (e.g., the monoazines, diazines, or triazines) may be the terminal hydrazine 18. For example, the electron withdrawing group may be a monoazine substituted with one hydrazine or two hydrazines. As described in more detail with respect to FIG. 3, at least one of the functional groups attached to the electron withdrawing group may be an alkyl group of the molecular chain portion 26.

In some embodiments, the electron withdrawing group may be an amide. As such, the terminal hydrazine 18 may be bonded to the carbon of the carbonyl group of the amide. In some embodiments, the linker 14 may include one or more intervening carbons between the carbonyl group and the terminal hydrazine 18. In embodiment when the linker 14 includes additional electron withdrawing groups, the additional electron withdrawing group may be disposed between the terminal hydrazine 18 and the amide. Alternatively, the amide may be adjacent to the terminal hydrazine 18 and disposed between the terminal hydrazine 18 and the additional electron withdrawing group.

In some embodiments, the electron withdrawing group may be a phenyl group. In some embodiments, the phenyl group may be substituted with one or more functional groups, such as halides, carboxylic acids, esters, amines (e.g., primary, secondary, or tertiary amines). In some embodiments, the phenyl group may be substituted with at least one of the terminal hydrazines 18.

In some embodiments, the electron withdrawing group may include a combination of one or more of the functional groups described above. For example, the linker 14 may include both an azine and a phenyl, a sulphonyl and a phenyl, and amide and a phenyl, an amide and a pyridine, or other combinations of electron withdrawing groups known by one of ordinary skill the art. Further, each electron withdrawing group may be separated by 2, 1, or 0 intervening carbon atoms. Accordingly, the linker 14 may include terminal hydrazine 18 that is adjacent to a first electron withdrawing group. Additionally, the linker 14 may include a second electron withdrawing group, and the first electron withdrawing group may be disposed between the terminal hydrazine 18 and the second electron withdrawing group.

Table 1 shows non-limiting examples of linkers 14 including electron withdrawing groups. Table 1 also shows example conditions for capturing and releasing the biological material 22 with the biological material complex 10.

TABLE 1
Example linkers for the biological material complex.

| | | | Catching | Releasing |
|---|---|---|---|---|
| Example | Solid Support | Linker end groups | conditions | conditions |
| 1 | Silica bead | Triazine substituted with | 60° C., 16-24 | 80° C., pH 6, 24 |
| | | hydrazine | hours, pH 6, | hours, aniline, |
| | | | aniline | biotinoxoamine |
| | | | | or |
| | | | | methoxyamine |
| 2 | Silica bead | Amide-hydrazine | RT, pH 4.5, | RT, 8-24 |
| | | | 8-24 hours, | hours, catalyst, |
| | | | catalyst | biotinoxoamine |
| | | | | or |
| | | | | methoxyamine |
| 3 | Silica bead | Amide-hydrazine | 60° C., 24 | 60° C., 24 hours, |
| | | | hours, | catalyst, |
| | | | catalyst | biotinoxoamine |
| | | | | or |
| | | | | methoxyamine |
| 4 | Silica bead | Amide-hydrazone | RT, pH 4.5, | RT, 8-24 |
| | | | 8-24 hours, | hours, catalyst, |
| | | | catalyst | biotinoxoamine |
| | | | | or |
| | | | | methoxyamine |
| 5 | Ni-Columm | amide-pyridine- | RT, pH 6, 4 | RT, pH 6, 4-8 |
| | | hydrazine | hours, aniline | hours, aniline, |
| | | | | biotinoxoamine |
| | | | | or |
| | | | | methoxyamine |
| 6 | Polystyrene | Phenyl-sulfonyl- | RT, pH 4.5, | RT, pH 4.5, 24 |
| | | hydrazine | 24 hours | hours |

Table 1 shows 6 examples (e.g., Example 1, Example 2, Example 3, Example 4, Example 5, and Example 6) of biological material complex 10. In general, each of the Examples correspond to a solid substrate 12 with a linker 14 that includes an electron withdrawing group and a terminal hydrazine. In general, the column ‘Linker end groups’ lists the electron withdrawing group and terminal hydrazine 18. Additionally, Table 1 displays catching conditions and releasing conditions for each of the examples. It should be noted that the conditions show in Table 1 are meant to be non-limiting. In general, the conditions for capturing or releasing the biological material 22 may be between room temperature to 80° C., at a neutral pH (e.g., approximately pH 7) or a relatively acidic pH (e.g., less than pH 7, less than pH 6, less than pH 6, between pH 4 to pH 6.5, between pH 4 to pH 6, between pH4 to pH 5, approximately 4, approximately 4.5, approximately 5, approximately 5.5, approximately 6, or approximately 6.5). In some instances, capturing and/or releasing may be performed in the presence of a catalyst. The catalyst may be any suitable catalyst for facilitating the reaction between an aldehyde and a hydrazine or hydrazide, such as certain benzoic acid catalysts (e.g., amino-benzoic acids), amino benzenes, aromatic amines, amino methyl imidazoles, or a combination thereof. For example, the catalyst may be 2-amino-5-methoxybenzoic acid or aniline. In some embodiments, the catalyst may be biotinoxoamine. In some embodiments, one or more of the catalysts described herein may be used.

Turning to the specific examples show in Table 1, Example 1 corresponds to silica beads (e.g., the solid substrate 12) with an attached linker 14 with a triazine (e.g., the electron withdrawing group) and two terminal hydrazine groups. Example 1 captured the biological material 22 at 60° C., over a time period between 16 to 24 hours, at a pH 6, in the presence of aniline. Example 1 released the biological material 22 at 80° C., over a time period of approximately 24 hours, at pH 6, and in the presence of aniline and biotinoxoamine or methoxyamine.

Example 2 corresponds to silica beads (e.g., the solid substrate 12) with an attached linker 14 with an amide and a terminal hydrazine. Example 2 captured the biological material 22 at room temperature (e.g., 25° C., over a time period between 8 to 24 hours, at pH 4.5, and in the presence of an amino-benzoic acid. Example 2 released the biological material 22 at room temperature, over a time period between 8 to 24 hours, at neutral pH, and in the presence of an amino-benzoic acid catalyst and biotinoxoamine or methoxyamine.

Example 3 corresponds to silica beads (e.g., the solid substrate 12) with an attached linker 14 that is the same linker 14 as described with respect to Example 2. Example 3 captured the biological material 22 at 60° C., over a time period of approximately 24 hours, at neutral pH, and in the presence of an amino-benzoic acid catalyst. Example 3 released the biological material 22 at 60° C., over a time period of approximately 24 hours, at neutral pH, and in the presence of an amino-benzoic acid catalyst and biotinoxoamine or methoxyamine.

Example 4 corresponds to silica beads (e.g., the solid substrate 12) with a linker 14 with an amide and a terminal hydrazone. Example 4 captured the biological material 22 at room temperature, over a time period between 8 to 24 hours, at pH 4.5, and in the presence of an amino-benzoic acid catalyst. Example 4 released the biological material 22 at room temperature, over a time period between 8 to 24 hours, at neutral, and in the presence of an amino-benzoic acid catalyst and biotinoxoamine or methoxyamine.

Example 5 corresponds to a nickel column (e.g., the solid substrate 12) with an attached linker 14 having an azine and a terminal hydrazine. Additionally, the linker 14 includes a histidine tail (e.g., at the first end 16) that may facilitate binding to the nickel column. Example 5 captured the biological material 22 at room temperature, over a time period of approximately 4 hours, at pH 6, and in the presence of aniline. Example 5 released the biological material 22 at room temperature, over a time period between 4 to 8 hours, at pH 6, and in the presence of aniline biotinoxoamine or methoxyamine.

Example 6 corresponds to a polystyrene bead (e.g., the solid substrate 12) with an attached linker 14 with a sulfonyl and phenyl group adjacent to the terminal hydrazine. Example 6 captured the biological material 22 at room temperature, over a time period of approximately 24 hours, at pH4.5. Example 6 released the biological material 22 at room temperature, over a time period of approximately 24 hours, at pH 4.5.

In general, Example 1, Example 2, and Example 5 have a relatively high yield for capturing the biological material 22 (e.g., under the reactions conditions presented in Table 1), such as capturing 70% or greater, 80% or greater, 90% or greater, or 95% or greater, approximately 90%, approximately 95%, or approximately 100% of the provided or available biological material 22. In contrast, Example 3, Example 4, and Example 6 have a relatively low yield for capturing the biological material 22 (e.g., under the reaction conditions present in Table 1), such as capturing 20% or less, 10% or less, 5% or less, or less than 1% of the provided or available biological material. It is presently recognized that the linkers 14 that include a terminal hydrazine had relatively higher capture yields as compared to linkers 14 that include a terminal hydrazone. Although the nitrogen that performs a nucleophilic attack on an aldehyde of the biological material 22 may appear to be more sterically available, nonetheless the linkers 14 with a terminal hydrazine demonstrated more efficient capturing.

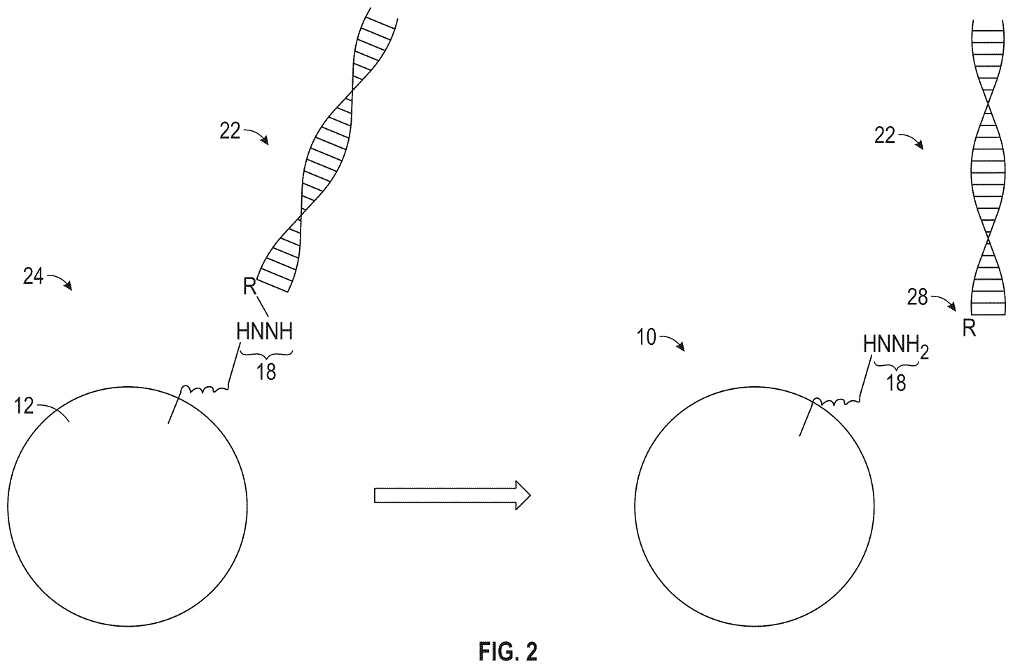

In some instances, it may be advantageous to selectively release previously captured biological material 22 from the linked biological material complex 24. FIG. 2 shows a schematic diagram of a process for selectively releasing a biological material 22 from the linked biological material complex 24. In general, FIG. 2 shows the reverse of capturing the biological material 22 as described in FIG. 1. In some embodiments, the biological material 22 may be released in substantially the same form as it was before being captured. That is, the biological material 22 may not be tagged, labeled, or otherwise include a chemical modification. However, at least in some instances, the biological material 22 may be tagged based on the selection of the R group 28. For example, and as discussed in more detail with respect to FIG. 4, it is presently recognized that when R is an aldehyde, the reaction between the aldehyde and the terminal hydrazine 18 may result in an oxime functional group disposed on an end of the biological material 22 that may act as a molecular tag, identifying the biological material 22 that was capture and released using the biological material complex 10.

FIG. 3 illustrates an example of a process 40 for producing the biological material complex 10 and the linker 14. At block 42, the process 40 includes providing a molecular chain 44 with an electron withdrawing species 46. As shown, the electron withdrawing species 46 is a triazine with three substituted chlorine groups. It is presently recognized that providing one or more substituted halogen groups onto the electron withdrawing species 46 may facilitate the linking the electronic withdrawing species 46 with the molecular chain portion 26, and forming the terminal hydrazine described in FIG. 1, to form the linker 14. In the illustrated embodiment, the molecular chain 44 includes an ethoxy silane group. In general, the ethoxy silane group may be any other suitable functional group for binding to a solid substrate 12 as described in FIG. 1. For example, in embodiments when the solid substrate 12 is a nickel column, the molecular chain 44 may include a histidine tail (e.g., 2, 3, 4, 5, or 6 histidines). As a further example, the linker may include silanes, thiols, carboxylic acids, phosphates, or other functional groups that may preferentially bind to a solid surface, such as glass, a silica bead, magnetic beads, metal particles, and the like.

It should be noted that the example process 40 is not limited to the molecular chain 44, the electron withdrawing species 46, or the linker 14 shown in FIG. 3. For example, the molecular chain 44 may be any molecular chain 44 discussed herein. For example, the molecular chain 44 may be an alkyl chain terminating with a phosphate group at a first end 16 described with respect to FIG. 1. As another non-limiting example, the electron withdrawing species 46 may be a monoazine, a diazine, a triazine, a sulphonyl, a phenyl, or other electron withdrawing groups, or any combination thereof of electron withdrawing groups.

In any case, the electron withdrawing species 46 and the molecular chain 44 react, at block 48, under suitable conditions to produce a molecular chain complex 50 that includes the molecular chain 44 linked to the electron withdrawing species 46. The conditions may include a suitable solvent (e.g., THF), at −10 In general, the molecular chain complex 50 is the molecular chain portion 26 before the molecular chain complex 50 is linked to the solid substrate 12 and the terminal hydrazine 18 is added. As one example of suitable reaction conditions for producing the molecular chain complex 50, the molecular chain complex 50 may be formed at −10° C., reacting over a time period of approximately 2 hours, and with tetrahydrofuran (THF) as a solvent. Further, the reaction may be performed in the presence of an alkaline species, such as trimethylamine to facilitate the coupling reaction between the molecular chain 44 and the electron withdrawing species 46 via the removal of chlorine as part of a tertiary amine salt.

At block 52, hydrazine is provided to the molecular chain complex 50 and reacted under suitable conditions to form the linker 14. As one example of suitable reaction conditions for producing the linker 14, the linker 14 may be formed by reacting hydrazine with the molecular chain complex 50 in the presence of an amine to facilitate removal of the chlorine, in a general similar manner as discussed in block 48. Further, ethanol may be added to the resulting product while maintaining a reaction temperature of approximately 0° C. Then, the product may be stirred for 2 hours while maintaining a reaction temperature of approximately 50° C., filtered, and isolated via chromatography, thereby providing the linker 14. At block 54, the solid substrate 12 may be provided to the linker 14 and reacted under suitable conditions to link the linker 14 to the solid substrate 12. For example, the suitable conditions may include utilizing a solvent, such as acetonitrile.

It should be noted that although the embodiment of the process 40 shown in FIG. 3 depicts the solid substrate 12 as being bead, the solid substrate 12 may be any solid surface as described herein. For example, the solid substrate 12 may be a flow cell, a column, a bead, and the like.

FIG. 4 shows an example process 60 for purifying biological material, which may generate tagged biological material 62. As discussed herein, the tag may include an oxime. At block 64, the process includes providing biological material 22 and the biological material complex 10. In this example, the biological material 22 is a nucleic acid 66. In particular, the nucleic acid 66 includes an aldehyde at the 5′ end of the nucleic acid 66. The 5′-aldehyde for oligonucleotides may be added during a coupling step (e.g., the last coupling step) in oligonucleotides synthesis on automated-oligosynthesizers. For enzymes, the aldehyde tag may be added through co-expression during enzyme fermentation.

In any case, at block 68, the biological material complex 10 reacts with the nucleic acid 66 under suitable conditions as discussed herein with respect to Table 1 to link the biological material 22 to the biological material complex 10, thereby forming the linked biological material complex 24. In some embodiments, it may be advantageous to link the biological material 22 to the biological material complex 10 at a pH less than 7, or from approximately 4 to approximately 6. As illustrated, the nucleic acid 66 is linked to the biological material complex 10 via a nitrogen of the terminal hydrazine (not shown).

At block 70, linked biological material complex 24 is subject to reaction conditions that release the tagged biological material 62 (e.g., labeled biological material, labeled nucleic acid, tagged nucleic acid). In this illustrated embodiment, the tagged biological material 62 includes an oxime functional group 72. It is presently recognized that providing an oxime functional group or other suitable functional groups in this manner may allow for tagging, labeling, or tracking of the tagged biological material 62. That is, the oxime functional group 72 or other suitable functional groups have a detectable spectroscopy signature (e.g., via Mass Spec, NMR, or the like) that enables a user to distinguish the tagged biological material 62 from other biological material (e.g., other nucleic acids).

At least in some instances, it may be desirable to release the biological material 22 without having the biological material 22 tagged with the oxime functional group 72. However, it may still be desirable to capture the biological material 22 via an aldehyde group at a 5′-end of the biological material 22 in an embodiment where the biological material 22 is a nucleic acid. To do so, it is presently recognized that it may be advantageous to insert a uracil (U) or 8-oxoG relatively close to (e.g., within 1, 2, 3, 4, or 5 oligonucleotides) 5′-side of the oligo to facilitate release of the biological material 22 while maintaining the ability of the hydrazine to capture the aldehyde. In such embodiments, the purified biological material released from the linked biological material complex, may not include a molecular signature, such as the oxime functional group 72. In embodiments where the molecular signature (e.g., the oxime functional group 72) is included on the tagged biological material 62, the molecular signature may be removed from the tagged biological material 62 enzymatically at the U or 8-oxoG position. In some embodiments, Uracil and 8-oxoG can be inserted consecutively at, for example, position 50 and position 100 for a 105 mer oligonucleotides, because Uracil and 8-oxoG may utilize different enzymes for cleavage. As such, this may provide release of two 50 mer oligonucleotides consecutively.

In some instances, it may be advantageous to wash the biological material complex 10 such that it may be reused. As discussed herein, washing the biological material complex 10 may generally involve rinsing with water and an acid and/or base. In any case, after washing the biological material complex 10, block 52 may be repeated to reintroduce one or more hydrazines to form the linker 14.

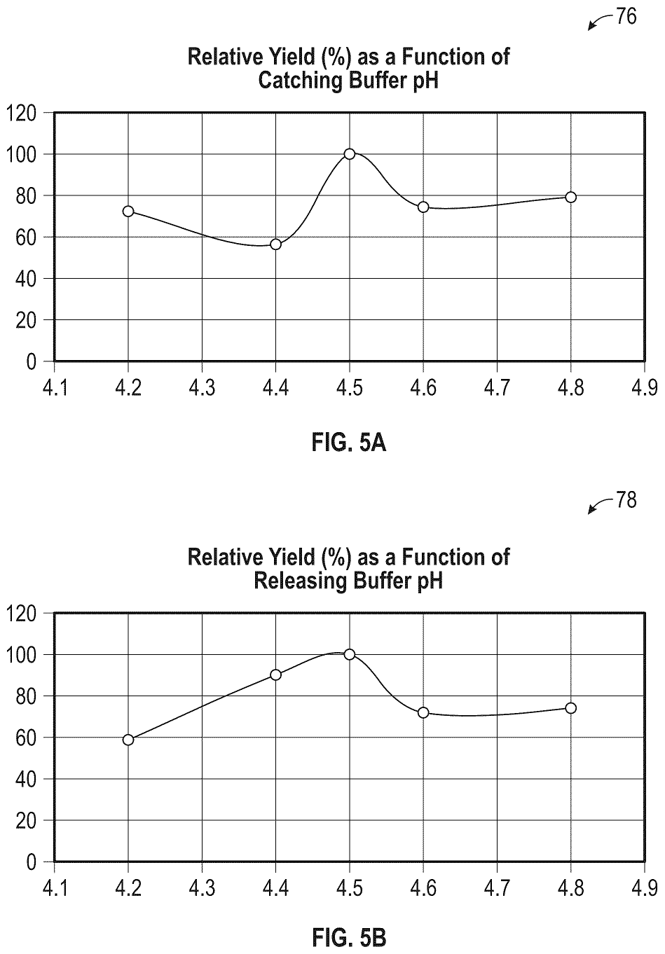

As discussed herein, the reaction conditions for capturing and/or releasing the biological material 22 using the biological material complex 10 (or the linked biological material complex 24) may involve performing the reaction within a particular pH range. FIG. 5A shows a graph 76 of reaction yield (y-axis) versus pH (x-axis) of the solution for capturing the biological material 22 with the biological material complex 10. In general, it is presently recognized that the yield of capturing the biological material 22 with the biological material complex 10 is dependent on pH. With the pH range shown in graph 76, there is at peak at approximately pH 4.5. Accordingly, it may be advantageous to capture the biological material 22 with the biological material complex 10 at pH 4.5 as compared to, for example, pH 4.4 or 4.6.

FIG. 5B shows a graph 78 of reaction yield (y-axis) versus pH (x-axis) of the solution for releasing the biological material 22 from the linked biological material complex 24. In a generally similar manner as described in FIG. 5A, it is presently recognized that the yield of releasing the biological material 22 from the linked biological material complex 24 is dependent on pH. With the pH range shown in graph 78, there is at peak at approximately pH 4.5. Accordingly, it may be advantageous to release the biological material 22 from the linked biological material complex 24 at pH 4.5 as compared to, for example, pH 4.4 or 4.6.

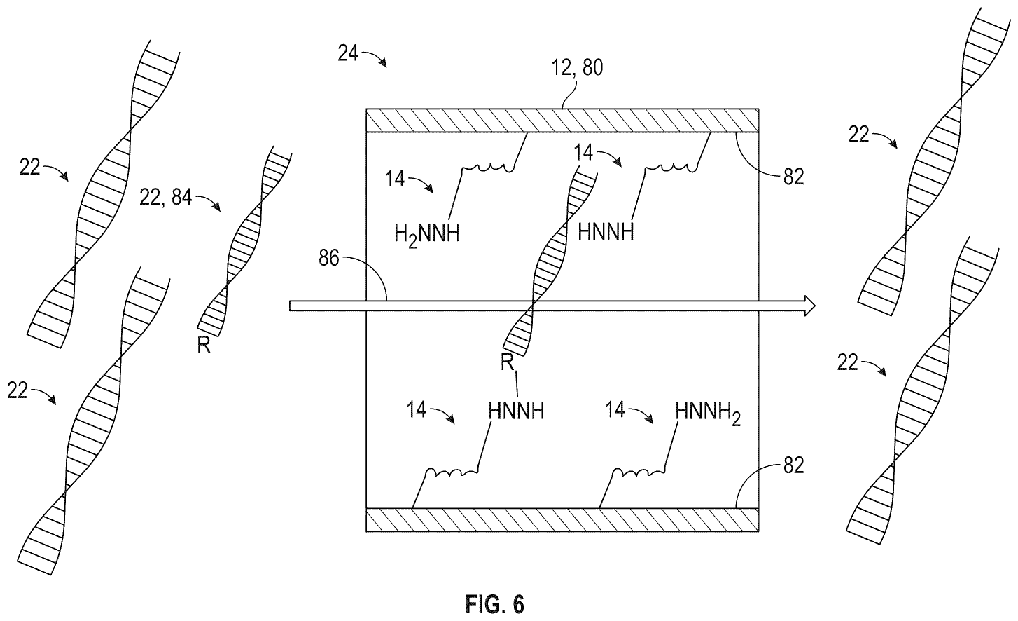

As discussed herein, it may be advantageous to form the biological material complex 10 on a column, such as nickel column or a silica gel column or the like. In particular, by forming the biological material complex 10 with a solid substrate 12 that is a column, the biological material complex 10 may be used to separate biological material from a solution by flowing the solution through the column. This is generally illustrated in FIG. 6. In particular, FIG. 6 shows a solid substrate 12 that is a nickel column 80. The nickel column 80 includes multiple linkers 14 bound to an inner surface 82 to the nickel column 80. Accordingly, when the biological material 22 flow through the column 80 (e.g., along the direction 86), biological material 22 that includes a functional group (i.e., biological material 84) that selectively binds to the linker 14 may captured onto the nickel column 80 (e.g., thereby forming a linker biological material complex 24). Accordingly, the capture biological material 22 may be removed from a solution of other biological material, and subsequently washed and released into solution.

It is presently recognized that it may be advantageous to tune the concentration of linkers 14 on the inner surface of the nickel column 80. In particular, tuning the concentration of the linkers 14 may provide techniques for controlling the amount of biological material 22 that is captured as the biological material passes through the nickel column 80.

Accordingly, the present disclosure relates to techniques for capturing, separating, labeling, or a combination thereof, biological material 22. The disclosed biological material complex 10 includes a terminal hydrazine that reacts with a function group (e.g., an aldehyde) on a biological material (e.g., an oligomer) to form a hydrazone that links the biological material complex 10 to the biological material 22, thereby forming the linked biological material complex 24. Once formed, the linked biological material complex 24 may be removed and/or transferred to another solution, washed, or otherwise isolated or purified. Then, the linked biological material complex 24 may be subject to releasing reaction conditions, thereby de-linking or releasing the biological material 22 from the linked biological material complex 24. As described herein, the released biological material may be a tagged biological material 62, having a molecule tag or label such as an oxime. The oxime may provide a useful molecular signature (e.g., detectable by optical spectroscopy techniques) for monitoring the tagged nucleic acid 62 in future reactions or use. It some instances, the, now, de-linked biological material complex 24 may be disposed of, which may reduce a likelihood of contamination that may result from repeated use. However, the de-linked biological material complex 24 may also be at least partially regenerated by providing additional hydrazine and reacting under suitable conditions described herein. Technical aspects of the present disclosure include, but are not limited to, facilitating purification of biological samples and/or concentration of the biological samples. Further, as compared to existing work flows, the disclosed biological material complex may reduce or prevent cross-contamination between samples by providing the biological material complex onto a relatively inexpensive solid substrate and/or reactivation of the biological material complex. Additionally, the disclosed techniques may be used to provide multiple active enzymes to perform multiple different functions (e.g., proteinase K and polymerase).

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function) . . . ” or “step for (perform)ing (a function) . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A biological material complex comprising:
a solid surface; and
a linker coupled to the solid surface at a first end of the linker and a terminal hydrazine or hydrazide coupled to a second end of the linker.

2. The biological material complex of claim 1, wherein the solid surface comprises polystyrene beads.

3. The biological material complex of claim 1, wherein the solid surface comprises silica beads.

4. The biological material complex of claim 1, wherein the solid surface comprises a nickel column.

5. The biological material complex of claim 1, wherein the linker comprises an electron withdrawing group adjacent to the terminal hydrazine.

6. The biological material complex of claim 5, wherein the electron withdrawing group comprises an azine, an amide, a phenyl, a sulfonyl, or a combination thereof.

7. The biological material complex of claim 6, wherein the electron withdrawing group comprises an azine, and wherein the azine comprises a monoazine, a diazine, or a triazine.

8. A method of preparing a biological material complex, comprising:
hybridizing a first end of a linker to a solid surface to form a biological material substrate, wherein the linker comprises a terminal hydrazine at a second end of the linker;
providing a biological material to a volume including the biological material substrate, wherein the biological material substrate comprises a terminal aldehyde; and
linking the biological material and the biological material substrate via a reaction between the terminal hydrazine and the terminal aldehyde.

9. The method of claim 8, wherein the biological material comprises an oligonucleotide.

10. The method of claim 8, comprising:
releasing a tagged biological material after linking the biological material and the biological material substrate, wherein the tagged biological material comprises the biological material and a molecular tag.

11. The method of claim 10, wherein the molecular tag comprises an oxime functional group.

12. The method of claim 8, wherein the linker comprises an electron withdrawing group that is adjacent to the terminal hydrazine.

13. The method of claim 12, wherein the electron withdrawing group comprises an azine, an amide, a phenyl, a sulfonyl, or a combination thereof.

14. The method of claim 13, wherein the solid surface is a nickel column, a flow cell, or a bead chip.

15. The method of claim 8, comprising washing the substrate containing the linked biological material and the biological material substrate.

16. The method of claim 8, comprising linking the biological material and the biological material substrate at a pH less than 7.

17. The method of claim 8, comprising linking the biological material and the biological material substrate at a pH from approximately 4 to approximately 6.

18. The method of claim 8, comprising linking the biological material and the biological material substrate in the presence of a catalyst.

19. A linked biological material complex, comprising:
a solid surface;
a linker coupled to the solid surface at a first end of the linker and a terminal hydrazine coupled to a second end of the linker, and wherein the linker comprises an electron withdrawing group; and
a biological material coupled to the second end of the linker via a hydrazone, wherein the hydrazone is adjacent to the electron withdrawing group.

20. The linked biological material complex of claim 19, wherein the electron withdrawing group comprises an azine, an amide, a phenyl, a sulfonyl, or a combination thereof.
